(19) United States
(12) Patent Application Publication
(10) Pub. No.: US 2018/0289890 A1
(43) Pub. Date: Oct. 11, 2018

(54) VOLUME ADJUSTMENT INFUSION SYSTEM AND METHOD

(71) Applicant: SEIRATHERM GMBH, Herzogenaurach (DE)

(72) Inventors: Matthias ROTH, Giggenhausen (DE); Thomas REICHTHALHAMMER, Polling (DE)

(73) Assignee: SEIRATHERM GMBH, Herzogenaurach (DE)

(21) Appl. No.: 15/764,641

(22) PCT Filed: Sep. 29, 2016
(86) PCT No.: PCT/EP2016/073322

§ 371 Date: Mar. 29, 2018

(30) Foreign Application Priority Data
Sep. 30, 2015 (EP) 15187808.9

(51) Int. Cl. A61M 5/168; A61F 7/12; A61M 5/44; A61F 7/00; A61M 5/172

(52) U.S. Cl. CPC A61M 5/16804; A61F 2007/0086; A61M 5/44; A61F 7/0085; A61M 5/1723; A61M 5/16895; A61M 2205/3393; A61M 2205/3389; A61M 2205/3303; A61M 2205/332; A61M 2205/3365; A61M 2205/3375; A61M 2205/3344; A61M 2205/3317; A61M 2205/3306; A61M 2230/04; A61M 2230/30; A61M 2205/3368; A61M 2230/42; A61F 7/12

(57) ABSTRACT

The present invention is directed to an adjustment device and method for adjusting the volume of one or more medical infusion fluids. This device can comprise any of at least one first determining unit adapted to measure and/or determine the volume of the medical infusion fluid flowing through a delivery duct and adapted to provide a respective first signal, at least one second determining unit adapted to measure and/or determine the volume and/or weight of at least one released body fluid and/or a physiological parameter and further adapted to provide a respective second signal and at least one volume controlling unit adapted to control the flow of the medical infusion fluid through the delivery duct on the basis of the first and the second signals.

### FIELD

The invention is directed to an adjustment device and method for adjusting the volume of medical infusion fluids. It can be used for any medical infusion purposes and for fever-treatment, normothermia and also hypothermia applications implementing infusions.

### BACKGROUND

Hypothermia is usually called a condition in which the body's core temperature drops below that required for normal metabolism and body functions. This is generally considered to be less than 35.0° C. (95.0° F.). Characteristic symptoms depend on the temperature. Targeted temperature management (TTM), previously known as therapeutic hypothermia or protective hypothermia is an active treatment that tries to achieve and maintain a specific body temperature in a person for a specific duration of time in an effort to improve health outcomes. This is done in an attempt to reduce the risk of tissue injury from lack of blood flow. Periods of poor blood flow may be due to cardiac arrest or the blockage of an artery by a clot such as may occur in stroke. Targeted temperature management improves survival and brain function following resuscitation from cardiac arrest. Evidence supports its use following ROSC (return of spontaneous circulation) after cardiac arrest. Targeted temperature management following a traumatic brain injury has some benefits. It can be used as neuroprotection. This therapy applied at the time of injury for a fixed duration of time can lessen the impact of a primary brain injury. Also, targeted temperature management can be aimed at lowering intracranial pressure (ICP) directly or indirectly contributing to reducing a secondary injury. ICP is the pressure inside a brain. Due to rigid intracranial vault, the volume inside the brain is constant. Intracranial tissue compartments are normally in a state of volume equilibrium. Hence, high ICP represents the force required to displace blood and cerebrospinal fluid from the intracranial space in order to accommodate new volume. ICP monitoring can also be used to guide medical and surgical interventions and to detect life-threatening imminent herniation. ICP is also dependent on the relationship between blood pressure and cerebral blood flow.

Targeted temperature management can advantageously prevent a brain injury by other methods including decreasing the brain's oxygen demand, reducing the proportion of neurotransmitters like glutamate, as well as reducing free radicals that might damage the brain. The lowering of body temperature may be accomplished by many means including the use of cooling blankets, cooling helmets, cooling catheters, ice packs and ice water lavage.

Medical events that targeted temperature management may effectively treat fall into five primary categories: neonatal encephalopathy, cardiac arrest, ischemic stroke, traumatic brain or spinal cord injury without fever, and any fever, e.g., neurogenic fever following brain trauma.

Document U.S. Pat. No. 7,896,834 B2 disdoses a pump system selectably controlling the temperature, flow rate, flow volume, and flow pressure of a fluid being infused into a patient's body. The apparatus comprises means for delivering a predetermined volume or halting device operation when an excessive volume has been infused.

Document U.S. Pat. No. 8,672,884 B2 discloses methods for introducing fluids into a body cavity for hypothermic treatment. In one embodiment of the invention, at least one of the rate or volume of infusate is configured to increase a mean patient blood pressure. In another embodiment, the infusion parameter is at least one of a flow rate, a pressure, a total infused volume, an inflow duty cycle or a hypothermic solution temperature.

The above mentioned prior art documents disclose methods of controlling the total fluid volume of the patient solely considering infusion volume and removal volume. However, there are further fluid volumes flowing out of the patient. These volumes can comprise, for example, emiction (urine), exhalation, evaporation, transpiration, and blood losses. The total fluid volume of the patient is likely to be different from the calculation disclosed in prior art. There is a risk of falling below the optimal fluid volume during surgery or temperature treatment.

### SUMMARY OF THE INVENTION

The problem underlying the present invention is how to provide an adjustment device and method for adjusting the volume of medical infusion fluids.

The problem is solved by the subject matter of the present invention exemplified by the description and the claims.

The inventors have developed a new and improved adjustment device and methods for adjusting the temperature and volume of a medical fluid such as an infusion liquid, which are able to maintain a desired targeted volume balance. The new device incorporates means for measuring and monitoring volume or weight of one or more bodily fluids from the patient, in particular emiction fluid (urine) and in some embodiments also other fluids discharged from the body, such as through perspiration, defecation, and blood loss. Through this invention, it is possible to improve the administration of a medical fluid such as an infusion fluid, in particular for the purpose of providing cooling of the patient, by ensuring optimal flow of the medical fluid taking into account the desired effect in terms of desired delivery volume and desired effect on body temperature, and an optimal volume balance of hydration of the patient.

The infusion fluid can be any among known fluids such as blood/blood derivates, pharmacological fluids, nutritional fluids, and fluid infusion systems and/or an infusion system for infusing, e.g., saline or other balanced fluids like ringer's solution. Also the kind, shape, material and volume can vary.

The present invention can be directed to an adjustment device for adjusting the volume of one or more medical infusion fluids. This device can comprise any of at least one first determining unit adapted to measure and/or determine the volume of the medical infusion fluid flowing through a delivery duct and adapted to provide a respective first signal, at least one second determining unit adapted to measure and/or determine the volume and/or weight of at least one released body fluid and/or a physiological parameter and further adapted to provide a respective second signal, and at least one volume controlling unit adapted to control the flow of the medical infusion fluid through the delivery duct on the basis of the first and the second signals. The term determining means can comprise any exact, estimated, direct, indirect measuring or modeling on the basis of other values.

The second determining unit can be adapted to measure and/or determine the volume and/or weight of at least one released body fluid selected from urine, sweat, wound liquid, blood, breath vapors, evaporation and/or liquid content of stools. The second determining unit may be used for determining or measuring just one of these or a number of them. The term second determining unit or second signal also represent further determining units or signals, such as third or fourth determining unit or third or fourth determining units etc. when more than a second parameter is determined.

The first determining unit and/or the second determining unit can comprise one or more weight, flow and/or volume measuring unit, preferably adapted to measure and to provide the respective first signal and/or second signal, respectively.

The first determining unit and/or the second determining unit can comprise at least one compartment or pouch or can be attached or integrated to it, the compartment or pouch for or later containing at least one fluid, such as infusion fluid or urine, and at least one associated flow rate sensor sensing the flow of one or more bodily fluid(s) out of and/or into the compartment or pouch and/or at least one associated volume sensor for measuring the volume of one or more infusion and/or bodily fluid(s) and/or one or at least one associated weight sensor weighing one or more infusion fluid and/or body fluid(s). Further, a transducer for providing the first signal and/or a transducer for providing the second signal can be provided. A transducer is meant to receive or derive a signal from the respective determination or measuring unit and to convert it into a signal which can be understood and/or processed by the volume controlling unit.

The adjustment device can comprise at least one compartment or pouch for at least one fluid, such as infusion fluid or urine, and the first determining unit and/or the second determining unit can comprise one force sensor for sensing the force, specifically the gravitational force, acting on the pouch and/or on the fluid.

The first determining unit and/or second determining unit can comprise an impeller sensor measuring flow rates, the impeller sensor comprising an impeller measuring flow rates by impeller rotations and a transducer for providing the first signal and/or a transducer for providing the second signal.

The first determining unit and/or second determining unit can comprise an ultrasound propagation sensor and a transducer for providing the first signal and/or a transducer for providing the second signal.

The first determining unit and/or the second determining unit can comprise a differential pressure sensor, preferably a multiphase flow meter, and a transducer for providing the first signal and/or a transducer for providing the second signal.

The first determining unit and/or the second determining unit can comprise at least one induction element adapted to determine the level of infusion fluid and/or of urine in a container or pouch and a transducer for providing the first signal and/or a transducer for providing the second signal.

The second determining unit can comprise a urine weight determining element comprising a capacitive element adapted to determine the level of urine in a container or pouch and a transducer for providing the second signal on the basis of the capacitive element. The capacitive element can be an elongate element which varies its capacity depending on the level of a liquid being adjacent to the elongate element. Usually, the elongate element is arranged in a vertical manner to any pouch or container.

The second determining unit can comprise an evaporation and/or a sweat measuring device, preferably adapted to be attached onto a skin of a patient, and further comprising an evaporation and/or a sweat permeable membrane and an evaporation and/or a sweat impermeable membrane on the opposite side, both defining an evaporation and/or a sweat reservoir, and a measuring device for measuring and/or determining the amount of evaporation and/or sweat in the evaporation and/or sweat reservoir, and a transducer for determining the total evaporation and/or sweat of a patient on the basis of the amount in the sweat reservoir and providing the second signal.

The second determining unit can comprise a hydric loss sensor adapted to be attached onto a skin of a patient and further comprising a swelling element being in direct contact to the skin and adapted to swell by absorption of the sweat and a measuring device measuring the increase in at least one dimension of the swelling element and a transducer for determining the total evaporation and/or sweat of a patient on the basis of the amount in the swelling element and providing the second signal.

The second determining unit can comprise an evaporation and/or a sweat measuring device comprising a signal supplier to generate an out-of-phase signal from a voltage signal obtained from a patient's skin and an in-phase signal from the voltage signal, a susceptance measurement unit to measure the susceptance of the voltage signal by synchronizing the voltage signal and the out-of-phase signal received from the signal supplier, a conductance measurement unit to measure the conductance of the voltage signal by synchronizing the voltage signal and the in-phase signal, and a transducer for determining the total sweat of the patient based on the measured conductance and skin moisture content information of the user based on the measured susceptance and providing the second signal.

The second determining unit can comprise at least one further hemodynamic sensor unit to measure and/or determine the hemodynamic status and a transducer for providing the second signal on the basis of the hemodynamic signal. The term hemodynamic sensor unit can be any sensor or plurality of sensors sensing hemodynamic factors, like blood pressure, blood composition, flow rates, flow volumes etc.

The hemodynamic sensor unit can comprise an optical hemodynamic sensor unit signal comprising a light source for transmitting light corresponding to first and second wavelengths through a blood perfused tissue of a patient and a light detector for generating optical signals corresponding to an intensity of the detected light at the first and second wavelengths.

The hemodynamic sensor unit can comprise at least one acoustical sensor for gaining acoustical data, preferably comprising a passive and/or active acoustical sensor, further circuitry for filtering and amplifying and digitizing the acoustical data and for providing the second signal.

The hemodynamic sensor unit can further comprise ECG electrodes to gain ECG data and preferably circuitry to compute and/or interpret the ECG data for providing the second signal.

The hemodynamic sensor unit can further comprise a vascular sensor for measuring and/or determining the vascular flow and/or heart time volume and/or the oxygen saturation of blood. The central venous pressure can be used as an input. The volume control unit can be adapted to measure the gradient (increasing or decreasing) of the central venous pressure. By means of it, the fluid flow can be controlled (reduced or raised) and the fluid balance can be achieved to keep the safe previously determined range, which for example allows to avoid hypovolemia and hypervolumia at the same time.

Static pressure indicators are sometimes not sensitive enough to predict hypovolemia or a patient's response to fluid administration. The flow-based parameters can be used. Sensors can detect and control units can control the urine output, the central venous pressure (CVP), the calibrated cardiac output (CO), the calibrated stroke volume (SV), the systemic vascular resistance (SVR), the pulse pressure variation (PPV), the stroke volume variation (SVV), the stroke volume index (SVI) and/or the respiratory variations in the plethysmographic waveform. Sensors can also detect and control units can control the volumetric parameters: the extravascular lung water (EVLW), the pulmonary vascular permeability index, the global end diastolic volume (GEDV), and/or the global ejection fraction.

Using urine output can lead to late detection of hypervolemia with no specific relation to volemia. The cardiac output, which is blood pumped from the heart in liters/min, can have no single absolute cardiac output target and can be highly invasive. The stroke volume is blood ejected from left ventricle per beat. The SVV is a variation in arterial pulsations caused by volume changes during positive pressure ventilation. SVV of more than 15% may indicate hypovolemia. The PPV, SVV, and respiratory variations can require mechanical ventilation, a tidal volume of at least 6 mL/kg, the patient to have no arrhythmia, and a closed chest condition. The respiratory variations method can be very sensitive to a vasomotor tone as well. The central venous pressure, which is the blood pressure in the venae cavae, near the right atrium of the heart can be invasive, but easy to monitor. It can be used with the cardiac output together as an input, and that can provide better indication of fluid responsiveness and a means of verifying that is beneficial to the patient's status.

Using continuous venous oximetry (ScvO2) monitoring, which is a real-time indicator of the balance between oxygen delivery (DO2) and oxygen consumption (VO2) can allow to monitor the brain's oxygen demand, preventing a brain injury. The central venous oxygen saturation, which is an assessment of balance between DO2 and VO2 can be used. Lower values indicate increased oxygen extraction or decreased delivery. Higher levels are seen with impaired oxygen utilization and lower extraction.

The volume control unit can be adapted to increase flow of infusion fluid upon measurement or determination of reduced hemodynamic action by the hemodynamic sensor unit.

The volume controlling unit can be adapted to receive and/or compute a pre-selected fluid balance level and to keep the fluid balance level on the basis of the first and the second signals.

The adjustment device can further comprise a fluid temperature unit for measuring and/or controlling the temperature of the medical infusion fluid and/or the temperature of an entity to be cooled and to deliver a third signal, and the volume controlling unit is configured to control the flow of the medical fluid, additionally on the basis of the third signal in order to adjust the temperature and/or the flow volume of the medical infusion fluid.

The second determining unit(s) can further comprise one or more sensor(s) for measuring at least one of temperature and/or humidity and to deliver a respective second signal on the basis of temperature and/or humidity.

Moreover, a temperature input device being connectable to a temperature sensor to monitor the temperature, preferably of a patient, the input device configured to receive a body temperature signal from the temperature sensor and to submit the signal to the volume controlling unit, wherein the volume controlling unit is configured to control the flow of the medical infusion liquid, additionally on the basis of the body temperature signal.

The volume controlling unit is adapted to determine normal and abnormal conditions and to further control the delivery of any medicament for compensating abnormal conditions.

The invention is also directed to a hypothermia system for inducing hypothermia with an adjustment device according to any one of the preceding claims. The invention can also be directed to a normothermia or hyperthermia system for controlling and/or stabilizing temperature.

The present invention provides an improved device for controlling and managing administration of infusion fluid that takes account of the electrolytic balance requirements of the human body. Thus, the device of the invention regulates and monitors overall electrolytic components administered to a patient and provides recommendations for type of infusion fluid during continued temperature therapy and/or directly controls the type of infusion fluid being administered in order to optimize the electrolyte content of administered infusion fluid.

The term electrolyte refers to a substance whose components dissociate in solution into positively and negatively charged ions (cation and anion) and thus the term electrolytic component refers to any such component of an electrolyte.

Infusion fluid refers to any fluid administered intravenously to a patient. Substances that may be infused intravenously include volume expanders, blood-based products, blood substitutes, medications and nutrition. Infusion solutions can be broadly divided in crystalloid and colloid solutions. Crystalloids are aqueous solutions of mineral salts or other water-soluble small molecules that readily diffuse across semi-permeable membranes. Colloids contain larger colloid molecules, such as but not limited to albumin, other blood proteins, gelatin, etc. that do not freely diffuse across a semi-permeable membrane (thus, blood is a colloid.) Accordingly, infusion solution as defined herein include, but are not limited to both crystalline solutions such as saline solutions or other type of conventional IV solution (such as but not limited to those examples shown in Table 1), dissolved drug or the like, and any type of colloid solution as well, administered to a patient via intravenous infusion.

Some common infusion fluids are defined below in a non-limiting list.

TABLE 1

| | Na⁺ | K⁺ | Ca⁺ | Mg⁺ | Cl⁻ | Lactate− | Acetate− | Osmolarity |
|---|---|---|---|---|---|---|---|---|
| | mmol/L | mmol/L | mmol/L | mmol/L | mmol/L | mmol/L | mmol/L | (mOsm/L) |
| NaCl | 154 | — | — | — | 154 | — | — | 308 |
| 0.9% | | | | | | | | |
| Ringer's | 147 | 4.0 | 2.3 | — | 156 | — | — | 309 |
| soln | | | | | | | | |
| Ringer- | 125-134 | 4.0-5.4 | 0.9-2.0 | — | 106-117 | 25-31 | — | 262-293 |
| lactate | | | | | | | | |
| soln | | | | | | | | |
| Ringer- | 130 | 5.4 | 0.9 | 1.0 | 112 | — | 27 | 276 |
| acetate | | | | | | | | |
| soln. | | | | | | | | |

It is advantageous that the device be configured to minimize shivering of the patient. Shivering is a normal reflex reaction of the body to feeling cold, triggered to maintain homeostasis. Skeletal muscles begin to shake in small movements, creating warmth by expending energy. Thus, shivering will counteract the desired effect of hypothermia treatment in addition to being uncomfortable to the patient. In an embodiment of the invention, the control unit of the device is configured to receive input signals indicating levels of shivering and to provide output signals indicating one or more recommendations for therapy based on said received input signals, to counteract the shivering. In one such embodiment, the volume control unit can alter (reduce) the flow rate, and/or raise the temperature of the infusion fluid. In another embodiment, the volume control unit provides an output signal indicating a recommendation, or a signal to a drug delivery device, that the patient be administered an anti-shivering medication, such as but not limited to a medication selected from opiates, tramadol, magnesium sulfate, α2-agonists, physostigmine, doxapram, methylphenidate, and/or 5-HT3 antagonists. In other embodiments, output signal indicate a recommendation that surface temperature of the patient be affected, such as through the use of blankets, heating pads, or the like.

Intracranial pressure (ICP) is the pressure inside the skull and thus in the brain tissue and cerebrospinal fluid (CSF). Increased intracranial pressure (ICP) is one of the major causes of secondary brain ischemia that accompanies a variety of pathological conditions, most notably, traumatic brain injury (TBI), stroke, and intracranial hemorrhages. In some embodiments, the control unit of the device is further configured to receive input signals indicating intracranial pressure (ICP) and, optionally, blood pressure of a patient, and to provide output signals indicating one or more recommendations for therapy based on said received input signals. Thus, the device can, in such embodiments, aid in the treatment of patients with elevated ICP. The input signals may be provided by a user, from an external computer system, or internally from a component of the device. The control unit may be configured to receive input signals indicating the level of intracranial pressure. ICP can be measured with invasive or non-invasive methods. Invasive methods normally require an insertion of an ICP sensor into the brain ventricle or parenchymal tissue. ICP can also be measured non-invasively. Several methods for noninvasive measuring of elevated ICP have been proposed: radiologic methods including computed tomography and magnetic resonance imaging, transcranial Doppler, electroencephalography power spectrum analysis, and the audiological and ophthalmological techniques. In one embodiment, the recommendation provided by the volume control unit comprises an instruction to administer ICP-reducing medication. As used herein, the term ICP-reducing medication is intended to mean any biologically active agent or drug or combination of agents or drugs that is administered to a patient for the purpose of reducing ICP. Any ICP-reducing agents can be used, such as agents commonly used in hyperosmolar therapy such as mannitol.

In a preferred embodiment, the control unit is configured to provide output signals to a drug delivery device adapted to administer said ICP-reducing medication, where the delivery device is not part of the overall device.

A drug delivery device includes any means for containing and releasing a drug, wherein the drug is released to a subject. The term “drug delivery device” refers to any means for containing and releasing a drug, wherein the drug is released into a subject. The means for containing is not limited to containment in a walled vessel, but may be any type of containment device, including non-injectable devices (pumps etc.) and injectable devices, including a gel, a viscous or semi-solid material, or even a liquid. Drug delivery devices may be inhaled, oral, transdermal, parenteral and suppository. Inhaled devices include gaseous, misting, emulsifying and nebulizing bronchial (including nasal) inhalers; oral includes mostly pills; whereas transdermal includes mostly patches. Parenteral includes injectable and non-injectable devices. Non-injectable devices may be “implants” or “non-injectable implants” and include e.g., pumps and solid biodegradable polymers. Injectable devices are split into bolus injections, that are injected and dissipate, releasing a drug all at once, and depots, that remain discrete at the site of injection, releasing drug over time. Depots include e.g., oils, gels, liquid polymers and non-polymers, and microspheres. Many drug delivery devices are described in Encyclopedia of Controlled Drug Delivery (1999), Edith Mathiowitz (Ed.), John Wiley & Sons, Inc. The term “drug” as used herein, refers to any substance meant to alter animal physiology. The term “dosage form” refers to a drug plus a drug delivery device. The term “formulation” (or “drug formulation”) means any drug together with a pharmaceutically acceptable excipient or carrier such as a solvent such as water, phosphate buffered saline or other acceptable substance. A formulation may contain a drug and other active agents. It may also contain an excipient, solvent or buffer or stabilizing agent.

In another preferred embodiment, the volume control unit is configured to provide output signals to a drug delivery device which is part of the overall device. In other words, the device according to present invention comprises a drug delivery device and wherein the control unit is configured to provide output signals to said drug delivery device. Such device may be semi-automated or automated, such that when the ICP is over a given, the control unit automatically provides an output signal to the drug delivery device adapted to deliver ICP-reducing medication to the patient without or with only minimal intervention of medical personnel.

The present invention also comprises all the steps to be performed or corresponding to any feature or hardware component described above or below or claimed.

All aspects of the present invention are adjusted to operate or be operated without a patient. According to one aspect of the present invention, the infusion fluid can be collected by a container or can be infused into a patient.

The preferred advantage of the present invention is to more effectively and more securely induce hypothermia. Thus, more individualized and a better adjusted flow of infusion fluids can be realized or a patient can be treated better.

### PREFERRED EMBODIMENTS

The present invention will become more fully understood from the description before and particularly below and the accompanying drawings that are given by way of illustration only and show and/or exemplify preferred aspects thereof, and wherein

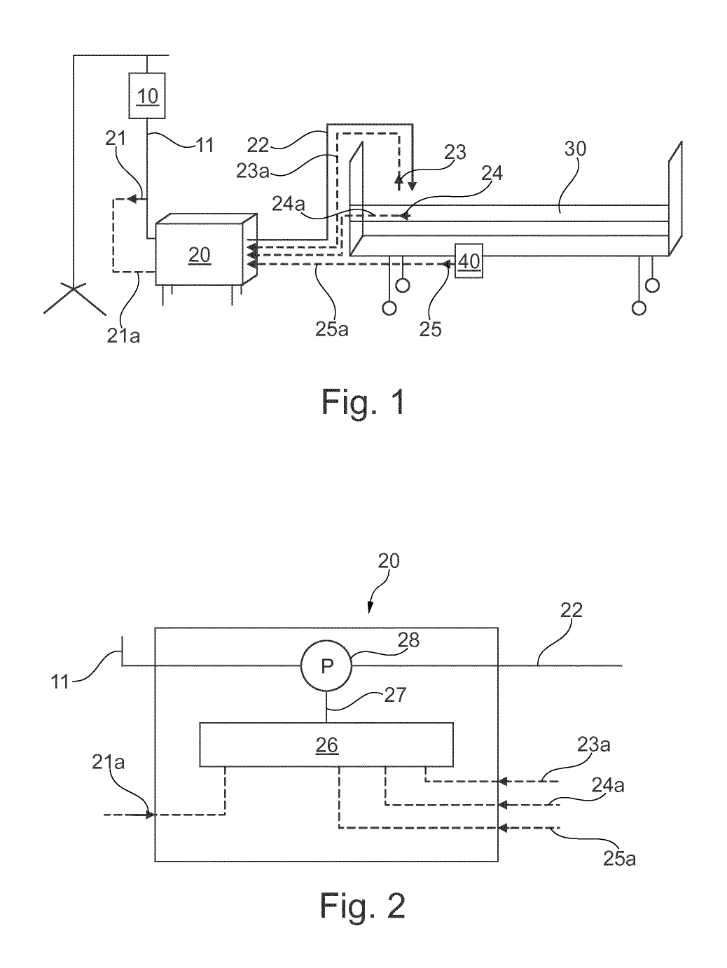

FIG. 1 is a principal sketch of a first preferred aspect of the present invention with at least one adjustment device in a potential environment; and

FIG. 2 shows a principle and view into an embodiment of an adjustment device according to the present invention.

FIG. 1 exemplifies one aspect of the present invention. A source 10 of a medical infusion fluid is shown, in the example shown, a typical bag 10 of infusion fluid is hung up on a supporting device often used in hospitals. Any other source, such as fixed and/or flexible containers of almost any shape and material is possible. From the bag 10, a duct 11 leads the infusion fluid towards a patient or container.

According to the prior art, the duct 11 is connected to or integral with the further duct 22 conveying the infusion fluid to the patient (not shown) in a bed 30. According to the present invention, a controlling unit or volume controlling unit 20 controls the infusion fluid on the basis of at least two or more parameters. In FIG. 1, it is exemplified that a sensor 21 can measure the volume or amount of fluid leaving the bag 10 and can feed this information or signal 21a into the controlling unit 20. Any dotted line in the figures exemplify a signal 21a, 23a, 24a, 25a in one or more respective feed-in or feedback line from a sensor 21, 23, 24, 25 to the controlling unit 20. Also, wireless data transmission such as Bluetooth or Wi-Fi or any other carrier can be chosen for this signal transfer. Further sensors 23, 24 and/or 25 with corresponding feedback lines can measure and/or determine other parameters and send them to the controlling unit 20. As mentioned before and below, the parameters can be body fluid(s) comprising a body fluid selected from urine, sweat, wound liquid, blood, evaporation, breath vapors, and/or liquid content of stools as well as other physiological parameters such as temperature, hemodynamic parameters, blood compositions, blood pressures etc.

FIG. 1 further exemplifies a pouch 40 for urine of a patient. In the embodiment shown, the volume and/or weight sensor 25 can sense the content in the pouch, and can deliver a respective signal 25a to the controlling unit 20.

FIG. 2 exemplifies a controlling unit 20 with the lines and/or ducts entering and/or leaving the controlling unit 20. In the embodiment shown, the duct 11 from the infusion fluid bag (not shown) enters the controlling unit 20. A pump 28 can then further convey and/or control amounts of volume to the exiting duct 22 towards a container or a patient (not shown). The pump can be any pump, such as a peristaltic pump or any other pump known.

The feed-in lines and/or the feedback lines feed their signals 21a,23a,24a,25a into a processing and/or a computing unit 26. As mentioned before, the signals 21a,23a,24a,25a can be conveyed by any means, such as by hard wiring or wireless technology or both.

In the computing unit 26, the appropriated control signal is generated, which controls the pump 28 via one or more control lines 27. The pump is then directed to either not convey infusion fluid or to convey infusion fluid and/or to modify flow rates in an appropriate amount from duct 11 to duct 22.

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components.

The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

The term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

It will be appreciated that variations to the foregoing embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” and the like, is merely intended to better illustrate the invention and does not indicate a limitation on the scope of the invention unless so claimed. Any steps described in the specification may be performed in any order or simultaneously, unless the context clearly indicates otherwise.

All of the features and/or steps disclosed in the specification can be combined in any combination, except for combinations where at least some of the features and/or steps are mutually exclusive. In particular, preferred features of the invention are applicable to all aspects of the invention and may be used in any combination.

1. An adjustment device for adjusting the volume of one or more medical infusion fluids, comprising
a. at least one first determining unit adapted to measure and/or determine the volume of the medical infusion fluid flowing through a delivery duct and adapted to provide a respective first signal,
b. at least one second determining unit adapted to measure and/or determine the volume and/or weight of at least one released body fluid and/or determine a physiological parameter and further adapted to provide a respective second signal, and
c. at least one volume controlling unit adapted to control the flow of the medical infusion fluid through the delivery duct on the basis of the first and the second signals.

2. The adjustment device according claim 1, wherein the second determining unit is adapted to measure and/or determine the volume and/or weight of at least one released body fluid selected from urine, sweat, wound liquid, blood, breath vapors, evaporation and/or liquid content of stools.

3. The adjustment device according to claim 1, wherein the first determining unit and/or the second determining unit comprises one or more weight, flow and/or volume measuring unit, preferably adapted to measure and to provide the respective first signal and/or second signal.

4. The adjustment device according to claim 3, wherein the first determining unit and/or the second determining unit comprises at least one compartment or pouch for at least one fluid, such as infusion fluid or urine, and at least one associated flow rate sensor sensing the flow of one or more infusion and/or bodily fluid(s) out of and/or into the compartment or pouch and/or at least one associated volume sensor for measuring the volume of one or more infusion and/or bodily fluid(s) and/or one or at least one associated weight sensor weighing one or more infusion fluid and/or body fluid(s) and a transducer for providing the first signal and/or a transducer for providing the second signal.

5. The adjustment device according to claim 1, wherein the adjustment device comprises at least one compartment or pouch for at least one fluid, such as infusion fluid or urine, and the first determining unit and/or the second determining unit comprises one force sensor for sensing the force, specifically the gravitational force, acting on the pouch and/or on the fluid.

6. The adjustment device according to claim 1, wherein the first determining unit and/or second determining unit comprises an impeller sensor measuring flow rates, the impeller sensor comprising an impeller measuring flow rates by impeller rotations and a transducer for providing the first signal and/or a transducer for providing the second signal.

7. The adjustment device according to claim 1, wherein the first determining unit and/or the second determining unit comprises an ultrasound propagation sensor and a transducer for providing the first signal and/or a transducer for providing the second signal.

8. The adjustment device according to claim 1, wherein the first determining unit and/or the second determining unit comprises a differential pressure sensor, preferably a multiphase flow meter, and a transducer for providing the first signal and/or a transducer for providing the second signal.

9. The adjustment device according to claim 1, wherein the first determining unit and/or the second determining unit comprises at least one induction element adapted to determine the level of infusion fluid and/or of urine in a container or pouch and a transducer for providing the first signal and/or a transducer for providing the second signal.

10. The adjustment device according to claim 1, wherein the second determining unit comprises a urine weight determining element comprising a capacitive element adapted to determine the level of urine in a container or pouch and a transducer for providing the second signal on the basis of the capacitive element.

11. The adjustment device according to claim 1, wherein the second determining unit comprises an evaporation and/or a sweat measuring device, preferably adapted to be attached onto a skin of a patient, and further comprising an evaporation and/or a sweat permeable membrane and an evaporation and/or a sweat impermeable membrane on the opposite side, both defining an evaporation and/or a sweat reservoir, and a measuring device for measuring and/or determining the amount of evaporation and/or sweat in the evaporation and/or sweat reservoir, and a transducer for determining the total evaporation and/or sweat of a patient on the basis of the amount in the sweat reservoir and providing the second signal.

12. The adjustment device according to claim 1, wherein the second determining unit comprises a hydric loss sensor adapted to be attached onto skin of a patient and further comprising a swelling element being in direct contact with the skin and adapted to swell by absorption of the sweat and a measuring device measuring the increase in at least one dimension of the swelling element and a transducer for determining the total evaporation and/or sweat of a patient on the basis of the amount in the swelling element and providing the second signal.

13. The adjustment device according to claim 1, wherein the second determining unit comprises an evaporation and/or a sweat measuring device comprising a signal supplier to generate an out-of-phase signal from a voltage signal obtained from a patient's skin and an in-phase signal from the voltage signal, a susceptance measurement unit to measure the susceptance of the voltage signal by synchronizing the voltage signal and the out-of-phase signal received from the signal supplier, a conductance measurement unit to measure the conductance of the voltage signal by synchronizing the voltage signal and the in-phase signal, and a transducer for determining the total sweat of the patient based on the measured conductance and skin moisture content information of the user based on the measured susceptance and providing the second signal.

14. The adjustment device according to claim 1, wherein the second determining unit comprises at least one further hemodynamic sensor unit to measure and/or determine the hemodynamic status and a transducer for providing the second signal on the basis of the hemodynamic signal.

15. The adjustment device according to claim 14, wherein the hemodynamic sensor unit comprises an optical hemodynamic sensor unit signal comprising a light source for transmitting light corresponding to first and second wavelengths through a blood perfused tissue of a patient and a light detector for generating optical signals corresponding to an intensity of the detected light at the first and second wavelengths.

16. The adjustment device according to claim 14, wherein the hemodynamic sensor unit comprises at least one acoustical sensor for gaining acoustical data, preferably comprising a passive and/or an active acoustical sensor, further circuitry for filtering and amplifying and digitizing the acoustical data, and for providing the second signal.

17. The adjustment device according to claim 14, wherein the hemodynamic sensor unit further comprises ECG electrodes to gain ECG data and preferably circuitry to compute and/or interpret the ECG data for providing the second signal.

18. The adjustment device according to claim 14, wherein the hemodynamic sensor unit further comprises a vascular sensor adapted to measure and/or determine the vascular flow and/or the heart time volume and/or the saturation of blood and/or the central venous pressure, and/or at least one, or any combination of the hemodynamic parameters comprising the calibrated cardiac output, the calibrated stroke volume, the systemic vascular resistance, the stroke volume variation, the stroke volume index, and/or at least one or any combination of the volumetric parameters comprising the extravascular lung water, the pulmonary vascular permeability index, the global end diastolic volume, the global ejection fraction.

19. The adjustment device according to claim 14, wherein the volume control unit is adapted to increase flow of infusion fluid upon measurement or determination of reduced hemodynamic action by the hemodynamic sensor unit.

20. The adjustment device according to claim 1, wherein the volume controlling unit is adapted to receive and/or compute a pre-selected fluid balance level and to keep the fluid balance level on the basis of the first and the second signals.

21. The adjustment device according to claim 1, wherein
a. the adjustment device further comprises a fluid temperature unit for measuring and/or controlling the temperature of the medical infusion fluid and/or the temperature of an entity to be cooled and to deliver a third signal, and
b. the volume controlling unit is configured to control the flow of the medical fluid additionally on the basis of the third signal in order to adjust the temperature and/or the flow volume of the medical infusion fluid (10).

22. The adjustment device according to claim 1, wherein the second determining unit(s) comprise one or more sensor(s) for measuring at least one of temperature and/or humidity and to deliver a respective second signal on the basis of temperature and/or humidity.

23. The adjustment device according to claim 1, further comprising a temperature input device being connectable to a temperature sensor to monitor the temperature, preferably of a patient, the input device configured to receive a body temperature signal from the temperature sensor and to submit the signal to the volume controlling unit, wherein the volume controlling unit is configured to control the flow of the medical infusion liquid additionally on the basis of the body temperature signal.

24. The adjustment device according to claim 1, wherein the volume controlling unit is adapted to determine normal and abnormal conditions and to further control the delivery of any medicament for compensating abnormal conditions.

25. The adjustment device according to claim 1, further comprising at least one third determining and or further determining unit(s) adapted to measure and/or determine the amount of electrolyte, shivering and/or intracranial pressure monitoring and adapted to provide at least one further respective third signal(s) and second controlling unit adapted to control the kind and dosage of the medical infusion fluid and/or any medicaments through the delivery duct on the basis of the third signal(s).

26. A hypothermia system for inducing hypothermia using the adjustment device according to claim 1.

27. A method of adjusting the volume of one or more medical infusion fluids, using the adjustment device according to claim 1, comprising the steps of
a. measuring and/or determining the volume of the medical infusion fluid flowing through a delivery duct and providing a respective first signal,
b. measuring and/or determining the volume and/or weight of at least one released body fluid and/or a physiological parameter and providing a respective second signal, and
c. controlling the flow of the medical infusion fluid through the delivery duct on the basis of the first and the second signals.

28. The method according to claim 27, comprising the further steps of receiving and/or computing a pre-selected fluid balance level and essentially keeping the fluid balance level on the basis of the first and the second signals.

29. The method according to 28, wherein the fluid balance level is kept with maximum 1 l, tolerance.

30. A method of treating a mammal comprising the method according to claim 27.
